(19) United States
(12) Patent Application Publication
(10) Pub. No.: US 2008/0009799 A1
(43) Pub. Date: Jan. 10, 2008

(54) Resilient protection sleeve for balloon catheter

(76) Inventor: Reinder N. Lap, Surhuizum (NL)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/480,057

(22) Filed: Jun. 30, 2006

(51) Int. Cl. A61M 5/00

(52) U.S. Cl. 604/171

(57) ABSTRACT

A sleeve may be used on a balloon catheter for several purposes, including protecting, forming and shaping the balloon, and also to reduce the balloon profile. A resilient protection sleeve may have a generally tubular structure, with one or more wings extending radially outward that act as resilient clamps. The tubular portion(s) extending between the wings may have an arcuate cylindrical shape, or any other tubular shape. The wings may also act as handles, to facilitate removal of the sleeve from the balloon catheter.

### BACKGROUND AND SUMMARY OF THE INVENTION

1. Technical Background

The present invention relates generally to medical devices, and more particularly to a resilient protection sleeve for balloon catheters.

2. Discussion

Balloon catheters are used in a variety of therapeutic applications. Structurally, many balloon catheters have a relatively long and flexible tubular shaft defining one or more passages or lumens, and an inflatable balloon attached near one end of the shaft. This end of the catheter where the balloon is located is customarily referred to as the “distal” end, while the other end is called the “proximal” end. The proximal end of the shaft is generally coupled to a hub, which defines a proximal inflation port and may define a proximal guidewire port. The proximal inflation port communicates with an inflation lumen defined by the shaft, which extends and is connected to the interior of the balloon, for the purpose of selectively inflating and deflating the balloon.

If the balloon catheter has a guidewire lumen for slidingly receiving a guidewire, the guidewire lumen will extend between a proximal guidewire port, and a distal guidewire port located at the distal end of the catheter. If the proximal guidewire port is defined at the hub, the resulting arrangement is referred to as “over-the-wire.” And if the proximal guidewire port is located at some intermediate point along the shaft, the resulting configuration is called “rapid-exchange.”

The balloon itself may have many different possible structures. For example, the balloon may have an inflatable central portion defining an inflated size, flanked by a pair of proximal and distal tapering conical portions, flanked by a pair of proximal and distal legs or collars. The proximal and distal collars may be affixed to the catheter shaft.

Examples of balloon catheters are shown in the following United States patents, all of which are commonly owned with the present invention: (i) U.S. Pat. No. 6,663,648, entitled “Balloon catheter with floating stiffener, and procedure,” issued to Trotta on Dec. 16, 2003; (ii) U.S. Pat. No. 5,824,173, entitled “Method for making a balloon catheter,” issued to Fontirroche et al. on Oct. 20, 1998; (iii) U.S. Pat. No. 5,820,594, entitled “Balloon catheter,” issued to Fontirroche et al. on Oct. 13, 1998; and (iv) U.S. Pat. No. 5,370,615, entitled “Balloon catheter for angioplasty,” issued to Johnson on Dec. 6, 1994.

During manufacture of a balloon catheter, the balloon component may be affixed to the shaft member(s), and then the balloon may be folded, pleated and wrapped around the shaft.

A protection sleeve may be slipped over the pleated balloon, at this point in the manufacturing process. This sleeve is used for not only protection, but also to form and hold a desired shape of the pleated balloon, and to reduce the initial profile of the balloon.

Prior protection sleeves were generally simple cylindrical tubes. To achieve the desired shape and small balloon profile, such sleeves may have a small diameter and a tight fit around the balloon and shaft. As a result, a prior protection sleeve may distort, crease, kink, twist or even cause damage to a balloon, which then increases the number of product rejects during manufacturing.

Accordingly, it would be desirable to provide a balloon protection sleeve with the properties of protection, shape forming and balloon profile reduction, while improving product yield rates during manufacturing.

By way of example, the present invention will be described in relation to balloon catheter angioplasty treatments and stenting procedures. However, it should be understood that the present invention relates to any resilient protection sleeve for a medical device or system including a balloon catheter, according to the present invention as recited in the following claims, and it is not otherwise limited to angioplasty, or stents, or any other feature that may be described in this description.

These and various other objects, advantages and features of the invention will become apparent from the following description and claims, when considered in conjunction with the appended drawings. The invention will be explained in greater detail below with reference to the attached drawings of a number of examples of embodiments of the present invention.

### BRIEF DESCRIPTION OF THE DRAWINGS

FIG. 1 is a perspective view of an example of a resilient protection sleeve according to the principles of the present invention;

FIG. 2 is an elevation view of the resilient protection sleeve of FIG. 1;

FIG. 3 is a perspective view of a resilient protection sleeve on a medical device system having a rapid-exchange arrangement, and a guidewire;

FIG. 4 is a perspective view of a resilient protection sleeve on a medical device system having an over-the-wire arrangement, and a guidewire;

FIG. 5 is a transverse cross-sectional view of a balloon catheter, in which the balloon has been pleated and wrapped around a portion of the catheter shaft;

FIG. 6 is a transverse cross-sectional view of a balloon catheter stent delivery system, in which a stent has been crimped around a pleated balloon;

FIG. 7 is a transverse cross-sectional view of the balloon catheter of FIG. 5, with a resilient protection sleeve; and

FIG. 8 is a transverse cross-sectional view of the balloon catheter stent delivery system of FIG. 6, with a resilient protection sleeve.

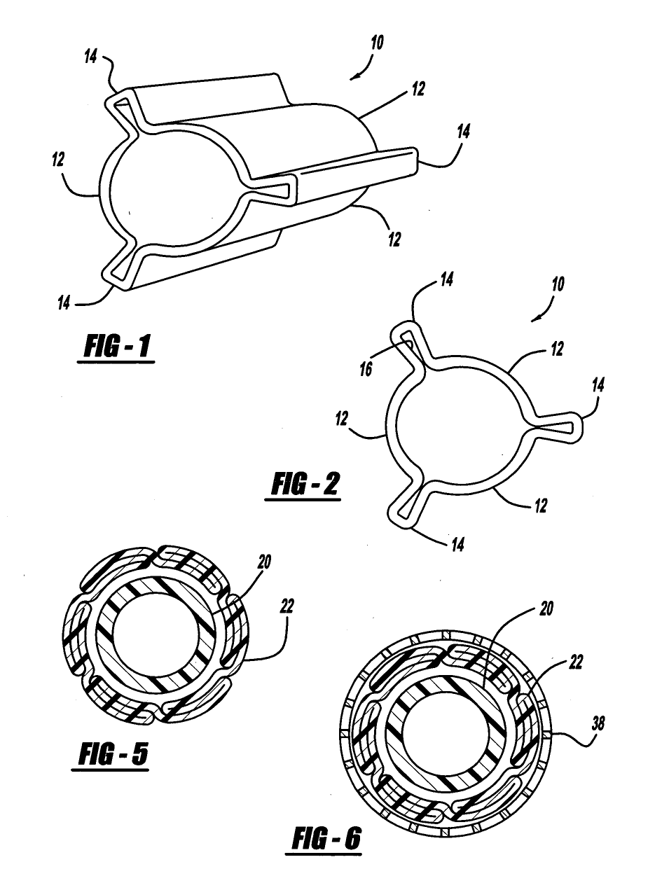

### DETAILED DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

A sleeve may be used on a balloon catheter for several purposes, including protecting, forming and shaping the balloon, and also to reduce the balloon profile.

A resilient protection sleeve has a generally tubular structure, with one or more tubular portions extending circumferentially between one or more wings that extend radially outward. The wings act as resilient clamps, to compress the tubular portions around a balloon of a catheter.

According to one example of the present invention, the drawings show a resilient protection sleeve 10 for use with a balloon catheter. The resilient protection sleeve 10 shown in the drawings has three tubular portions 12 and three wings 14. The tubular portions 12 each have an arcuate partial cylinder shape. The wings 14 each have a “U” shape that is narrowed at the junction between the the tubular portions 12 and the wings 14. Each wing extends radially outward, and each defines a channel 16.

The resilient protection sleeve 10 shown in the drawings is made of an integral and unitary piece of material, but it could be made of several components affixed together. Other possible arrangements of a resilient protection sleeve according to the principles of the present invention include providing a resilient protection sleeve having multiple layers of different materials, whether be coextrusion or any other suitable method. Also, it is possible to provide an inner surface of a resilient protection sleeve with a lubricious coating, for easy removal of the sleeve from the balloon catheter.

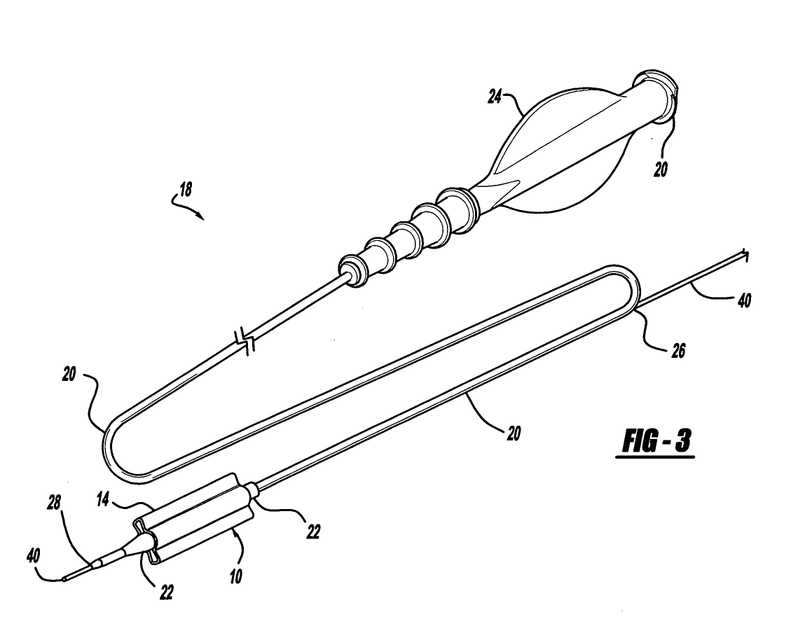

FIG. 3 shows a balloon catheter 18 having a catheter shaft 20 extending from a proximal end to a distal end, a balloon 22 affixed to the catheter shaft 20 near its distal end, and a hub 24 affixed to the catheter shaft 20 at its proximal end. The catheter shaft 20 defines a longitudinal axis, an inflation lumen and a guidewire lumen for receiving a guidewire 40, with the guidewire lumen extending between a proximal guidewire port 26 and a distal guidewire port 28, and the inflation lumen extending between a proximal port 30 defined by the hub 24 and an interior of the balloon 22.

The balloon 22 has a central inflatable portion between a proximal collar and a distal collar, the collars each being affixed to the catheter shaft 20. The balloon 22 is shown in an initial configuration, such that it has been deflated, pleated and wrapped around the catheter shaft 20.

In the initial configuration, the resilient protection sleeve compresses and protects the balloon, as the wings resiliently clamp the tubular portions of the sleeve around the balloon. When a physician prepares the balloon catheter for use in a medical treatment, the wings may also act as handles, to facilitate removal of the sleeve from the balloon catheter.

Dimensionally, the sleeve has a longitudinal length corresponding to a longitudinal length of the balloon, such that the sleeve protects the full length of the balloon.

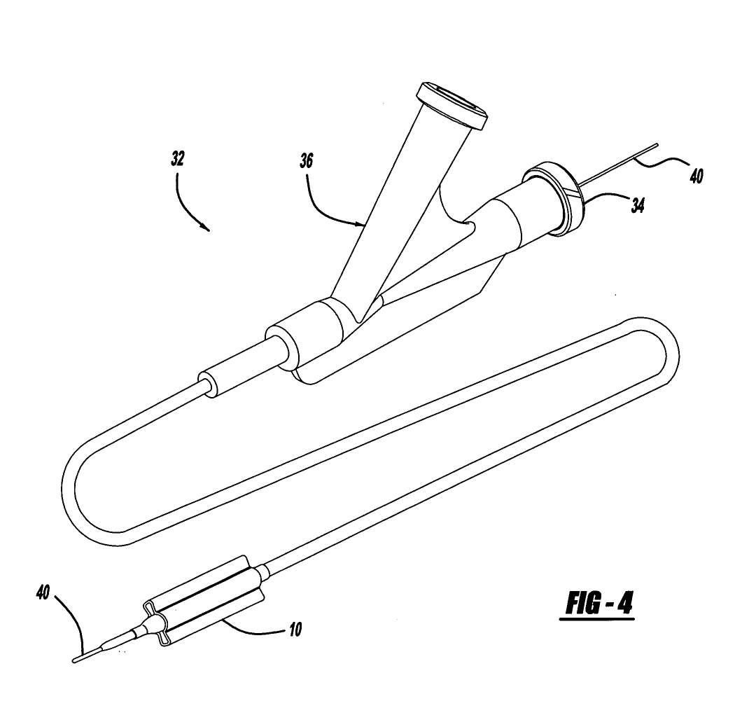

The balloon catheter of FIG. 3 has a rapid-exchange arrangement, in that the proximal guidewire port 26 is located at a position on the catheter shaft 20 between the balloon 22 and the hub 24. The balloon catheter 32 of FIG. 4 is similar, except that it has an over-the-wire arrangement, in that the proximal guidewire port 34 is defined by the proximal hub 36.

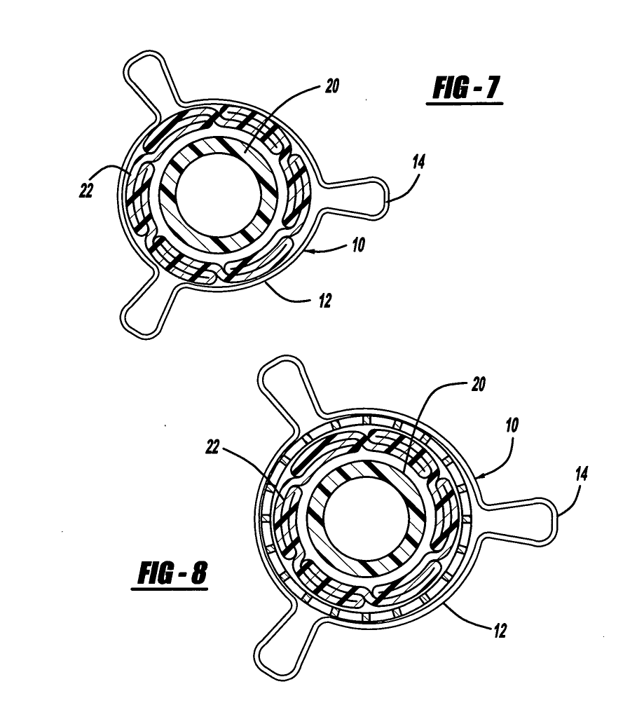

FIG. 5 shows a cross-section of a balloon 22 in an initial configuration, deflated, pleated and wrapped around a tubular member of a balloon catheter shaft 20. FIG. 7 is the same as FIG. 5, with the addition of a resilient protection sleeve 10.

Also, the balloon catheter systems of the present invention may include one or more additional medical device(s). For example, FIG. 6 shows a cross-section of a balloon catheter with a medical device, in this case a stent 38, crimped about the deflated, pleated and wrapped balloon 22 in an initial configuration. FIG. 8 is the same as FIG. 6, with the addition of a resilient protection sleeve 10.

The resilient protection sleeve may be made of any material having suitable properties, including polymers, including for example polycarbonates, polyamides, polyurethanes, nylons, polyethylenes, and polyesters. Also, any of the catheter components may be made of a multilayer construction or a coextrusion, or a blend or a block copolymer of such polymer materials.

It should be understood that an unlimited number of configuration for the present invention could be realized. The foregoing discussion describes merely exemplary embodiments illustrating the principles of the present invention, the scope of which is recited in the following claims. Those skilled in the art will readily recognize from the description, claims and drawings that numerous changes and modifications can be made without departing from the spirit and scope of the invention.

What is claimed is:

1. A balloon catheter system for medically treating a patient, comprising:
a balloon catheter having a catheter shaft extending from a proximal end to a distal end, a balloon affixed to the catheter shaft at or near its distal end, and a hub affixed to the catheter shaft at or near its proximal end;
wherein the shaft defines a longitudinal axis, an inflation lumen and a guidewire lumen, the guidewire lumen extending between a proximal guidewire port and a distal guidewire port, the inflation lumen extending between a proximal port defined by the hub and an interior of the balloon;
wherein the balloon has a central inflatable portion between a proximal collar and a distal collar, the collars each being affixed to the catheter shaft; the balloon in an initial configuration being deflated, pleated and wrapped around the catheter shaft; and
a resilient protective sleeve around the balloon, the sleeve having at least one tubular portion and at least one wing extending outward, each wing defining a channel and resiliently clamping the sleeve around the balloon, each tubular portion extending circumferentially between each wing, the sleeve having a longitudinal length corresponding to a longitudinal length of the balloon, such that the sleeve protects and compresses the balloon.

2. The balloon catheter system of claim 1, wherein the sleeve has three tubular portions and three wings.

3. The balloon catheter system of claim 1, wherein each tubular portion has the shape of a partial cylinder.

4. The balloon catheter system of claim 1, wherein each wing has a narrowed “U” shape.

5. The balloon catheter system of claim 1, wherein the sleeve is made of a polymer material.

6. The balloon catheter system of claim 1, wherein the sleeve is made of a single integral and unitary piece.

7. The balloon catheter system of claim 1, wherein the sleeve has multiple layers of different materials.

8. The balloon catheter system of claim 1, wherein an inner surface of the sleeve has a lubricious coating.

9. The balloon catheter system of claim 1, wherein the balloon catheter has an over-the-wire arrangement.

10. The balloon catheter system of claim 1, wherein the balloon catheter has a rapid-exchange arrangement.

11. The balloon catheter system of claim 1, further comprising a medical device, the medical device in an initial configuration being disposed between the balloon and the sleeve.

12. The balloon catheter system of claim 8, wherein the medical device is a stent.
